(19) United States
(12) Patent Application Publication
(10) Pub. No.: US 2014/0330258 A1
(43) Pub. Date: Nov. 6, 2014

(54) MEDICAL LASER APPARATUS

(71) Applicant: Lumenis Ltd., Yokneam Ilit (IL)

(72) Inventor: Yoni IGER, Haifa (IL)

(21) Appl. No.: 14/242,449

(22) Filed: Apr. 1, 2014

(30) Foreign Application Priority Data
Apr. 1, 2013 (GB) 1305881.3

(51) Int. Cl. A61B 18/20

(52) U.S. Cl. CPC A61B 18/203; A61B 2018/208
USPC 606/3; 606/9

(57) ABSTRACT

A medical apparatus comprising a first laser source operable to generate light at a first wavelength and supply a first laser light beam, a second laser source operable to generate laser light at a second wavelength and supply a second laser light beam, the first laser light beam being continuous or pulsed having a first, relatively long pulse duration, the second laser light beam being pulsed with a second, relatively short pulse duration, the apparatus being operable to supply the first laser light beam and subsequently the second laser light beam.

### SUMMARY OF THE INVENTION

The present invention relates to a medical laser apparatus and a method of operating a medical laser apparatus, particularly but not exclusively for cosmetic treatment of skin.

### BACKGROUND TO THE INVENTION

Non-ablative treatment of skin using lasers is an established technique. It has been used for treatment of a number of conditions, including striae, dyschromia and lesions in the skin such as age spots and melasma, and the treatment of wrinkles, acne scars and surgical scars.

In non-ablative treatment, a laser beam at a suitable wavelength and power is directed at a location of the skin, such that the skin surface is not removed or ablated but skin tissue is locally heated. The local heating causes coagulation of the tissue, creating a microscopic, demarcated and isolated, conical thermo-coagulation wound zone. An array of such coagulated zones is created across the skin area to be treated.

The wound coagulation zones cause collagenesis and remodelling of the skin, by releasing and activating different interleukins, heat shock proteins, growth factors and other wound healing mediators. The skin around each thermo-coagulation zone is undamaged, and fibroblasts migrate to the wounded zone and in particular are effective there in synthesis of collagen. The technique is advantageous as the stratum corneum, the top of the skin, remains intact and the skin itself is not ablated, reducing the risk of potential side effects and infection, and lowering patient discomfort.

### SUMMARY OF THE INVENTION

According to a first aspect of the invention there is provided a medical apparatus comprising a first laser source operable to generate light at a first wavelength and supply a first laser light beam, a second laser source operable to generate laser light at a second wavelength and supply a second laser light beam, the first laser light beam being continuous or pulsed having a first, relatively long pulse duration, the second laser light beam being pulsed with a second, relatively short pulse duration, the apparatus being operable to supply the first laser light beam and subsequently the second laser light beam.

The first laser light beam may have a diameter in the range of approximately 100 microns to 400 microns, and preferably in the range 110 to 120 microns.

The second laser light beam may have a diameter in the range of about 2 to 10 mm, and preferably about 2 to 5 mm.

The first wavelength may be in the range 1300 nm to 1600 nm, and preferably about 1565 nm, preferably such that the first laser beam has a desired coagulative effect.

The second wavelength may be in the range 500 to 1300 nm.

The apparatus may be operable to generate the second laser light beam with a pulse length in the range 1 to 100 ns, and preferable less than 10 ns, and most preferable 6 to 8 ns.

Alternatively the apparatus may be operable to generate the second laser light beam with a pulse length in the range 1 to 700 ps.

The apparatus may comprise a treatment head wherein at least the first laser light beam is supplied to the treatment head.

The treatment head may comprise a scanning device, the scanning device being operable to direct the first laser light beam to a plurality of locations within a treatment area.

The first laser source may comprise a fibre laser.

The second laser source may comprise a Q-switched laser.

According to a second aspect of the invention there is provided a method of directing laser light to a skin surface, the method comprising the steps of generating a first laser light beam having a first wavelength and direct the first laser light beam to an irradiation area, subsequently generating a second laser light beam having a second wavelength and directing the second laser light beam to an irradiation area, the first laser light beam being continuous or pulsed having a first, relatively long pulse duration, the second laser light beam being pulsed with a second, relatively short pulse duration.

The method may comprise a method of cosmetically treating an area of skin.

The first laser light beam may have a diameter in the range of approximately 100 microns to 400 microns, and preferably in the range 110 to 120 microns.

The second laser light beam may have a diameter in the range of about 2 to 10 mm, and preferably in the range 2 to 5 mm. The first wavelength may be in the range 1300 nm to 1600 nm, and preferably about 1565 nm, preferably such that the first laser beam has a desired coagulative effect.

The second wavelength may be in the range 500 to 1300 nm.

The second laser light beam may have a pulse length in the range 1 to 100 ns, and preferably less than 10 ns, and most preferably 6 to 8 ns.

The second laser light beam may have a pulse length in the range 1 to 700 ps.

The method may comprise directing the first laser light beam to a plurality of locations within a treatment area.

Generating the first laser light beam may comprise operating a fibre laser.

Generating the second laser light beam may comprise operating a Q-switched laser.

### BRIEF DESCRIPTION OF THE DRAWINGS

Embodiments of the invention will now be described by way of example only with reference to the accompanying drawings wherein;

FIG. 1a is a diagrammatic illustration of a first apparatus embodying the invention,

FIG. 1b is a diagrammatic illustration of a second apparatus embodying the invention,

FIG. 1c is a diagrammatic illustration of a third apparatus embodying the invention,

FIG. 2 is an illustrative cross-section through a dermis and epidermis during a first irradiation step,

FIG. 3 is a view of a plurality of alternative beam locations of the step of FIGS. 2, and

FIG. 4 is a view similar to FIG. 2 during a subsequent irradiation step.

### DETAILED DESCRIPTION

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated n the drawings. The invention is applicable to other embodiments or of being practiced or carried out in various ways. Also, it is to be understood that the phraseology and terminology employed herein is for the purpose of description and should not be regarded as limiting.

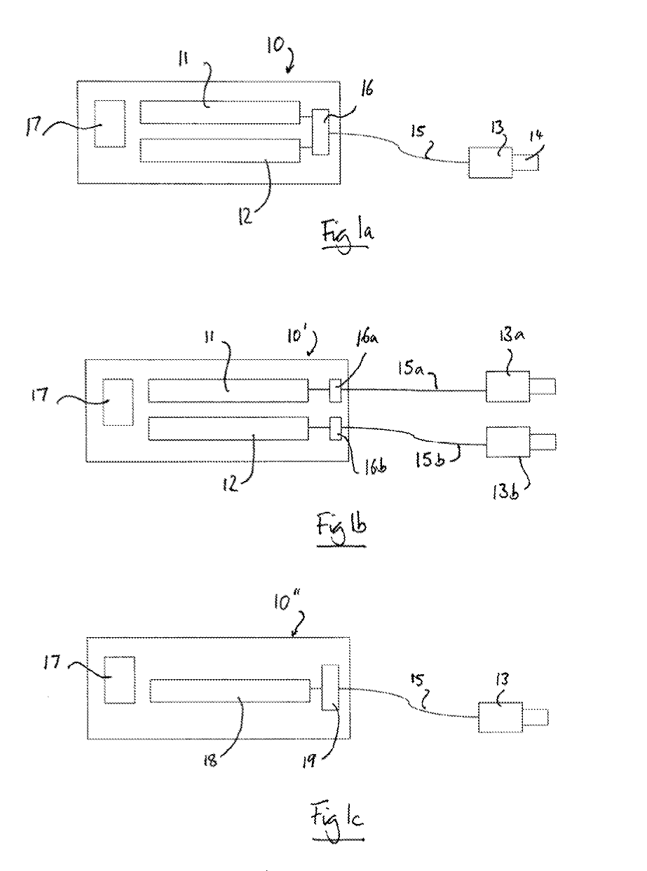

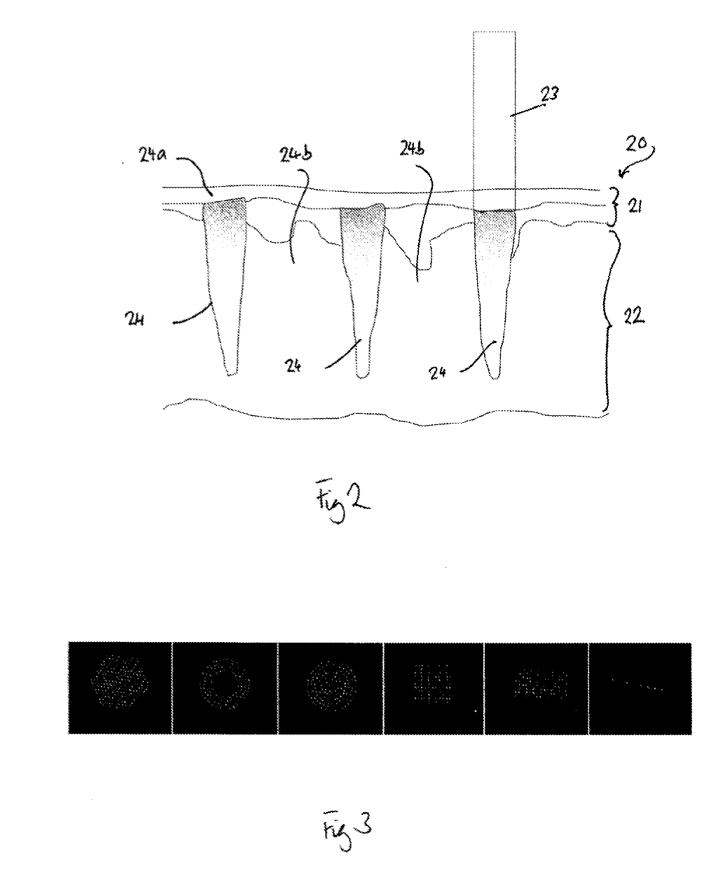

Referring now to FIG. 1a, a medical apparatus is illustrated generally at 10. The apparatus 10 comprises a first laser source 11, and a second laser source 12. In this description, ‘laser source’ is intended to refer to the laser cavity or resonant element, together with any required control elements and beam conditioning or modulation elements. A treatment head is provided shown at 13, in the present example with an adjustable end part 14. The treatment head 13 is linked to the laser sources by a suitable connection shown at 15, which may be an optical fibre connection, or an internally reflective arm, or any other suitable connection as desired. A beam direction apparatus 16 transmits a first laser beam from the first laser source or a second laser beam from the second laser source 12 to the connection 15 as desired. A controller 17 is provided to control the respective laser sources 11, 12 and provide beam power and characteristics as desired under the control of a suitable operator.

In an alternative configuration, as shown in FIG. 1b, the apparatus 10′ may have separate treatment heads 13a, 13b, connected to the first laser source 11, second laser source 12 respectively, by suitable connectors 15a, 15b and optical components 16a, 16b. In a further alternative configuration, apparatus 10″ comprises a single laser element 18 and beam modulation optics 19. By controlling the laser element 18 and modulation optics 19, in a first mode of operation the system may operate as a first laser source and generate the first laser beam, and in a second mode of operation operate as a second laser source and generate the second laser beam. The modulation optics 19 may be operable to provide frequency doubling or electro-optical manipulation in any other way to produce a second laser beam which is different than the first laser beam.

The first laser source 11 may have a wavelength in the range 1100 nm to 1600 nm, and in the present example comprises a fibre laser with an output wavelength of 1565 nm and a beam energy of 10 to 70 mJ. The first laser light beam from the first laser source 11 is either a continuous wave beam, or has a relatively long pulse length, of up to 0.5 s. The second laser source 12 comprises a Q-switched laser, in the present example a Q-switched 1064 nm Nd:YAG laser. Q-switching is a known technique in which the energy in a laser cavity is released in a very short, high-power pulse. In this example, the pulse length is in the range 1 to 100 ns, and preferably less than 10 ns and more preferably in the range 6 to 8 ns, although any other appropriate pulse length may be used as desired. If shorter pulse lengths are required, the second laser source 12 could be a picoseconds-laser source, which permits the pulse length to be in the range 1 to 700 ps. Nd:YAG lasers may be operated to obtain a desired wavelength, preferably in the range 500 to 1300 nm. Other laser types, such as an Er:YAG, thulium, holmium or other known solid state, fibre or gas lasers, may alternatively be used.

The adjustable head 14 of the treatment head 13 preferably comprises a scanning device operable to direct the first laser light beam to a plurality of locations over a desired area for treatment. In this example, the first laser light beam preferably has a diameter in the range 100 to 400 microns, and preferably about 110 to 120 microns. The beam is directed to a plurality of locations over a much larger area of skin as needed, for example having a diameter to the range 5 to 20 mm, such that the beam is directed to 50 to 500 spots per cm² of treated skin.

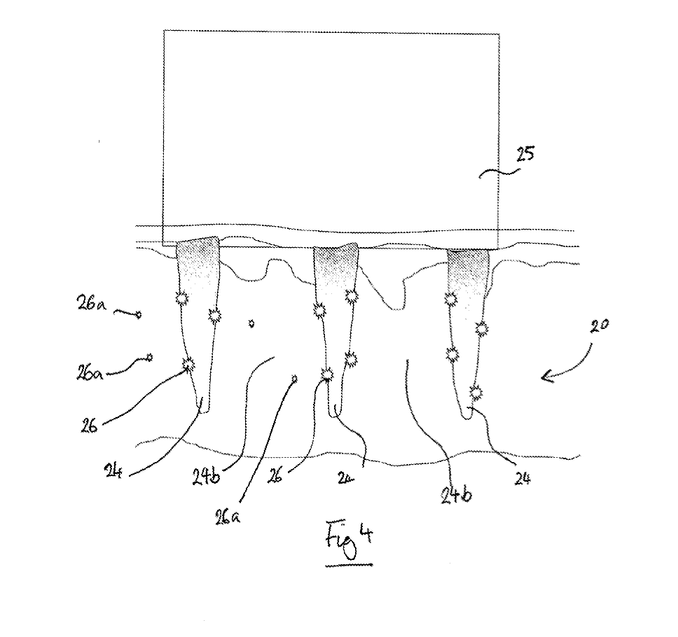

During operation of the first laser source 11, a skin section is generally shown at 20 in FIG. 2. The layers of the epidermis are generally shown at 21 and the dermis illustrated at 22. The first laser light beam is diagrammatically illustrated at 23. As shown at 24, in a first irradiation step the beam 23 is directed at the skin 20, where a local fractional volume of the skin is heated to coagulative level. The wavelength of the first laser beam 23 is selected to have a desired coagulative impact. The treated areas have a width which depends on the width of the first beam 23, in this example generally in the range 100 to 400 microns. The areas 24 extend into the dermis depending on the wavelength and power of the first laser light beam 23 and the length of time the area is irradiated, in this example up to 800 microns. As illustrated at 24a, the upper layer of the epidermis is undamaged or substantially undamaged, reducing the chance of microbial infections. As illustrated at 24b, the regions of skin 20 between the areas 24 are substantially unharmed and unaffected. The first laser light beam 23 may be directed to form an array of areas 24 in any suitable manner as required, depending on the area and condition to be treated. Suitable patterns are illustrated in FIG. 3, including hexagonal, annular, circular, square, rectangular and linear arrangements of areas 24.

In a subsequent irradiation step, a second laser light beam is generated from the second laser source 12 and directed to the treated area of skin 20 as shown in FIG. 4. In this example, it will be apparent that the second laser light beam 25 has a much larger diameter than the first laser light beam 23, for example, second laser light beam 25 may have a beam width of 1 to 10 mm, preferably 2 to 5 mm (note that the illustration in FIG. 4 is not to scale) and as such will be able to cover a portion of the array of areas 24 illustrated in FIG. 3, thus irradiating a plurality of areas 24 simultaneously. The pulsed second laser light beam 25 affects in particular the coagulated wound tissue of the zones 24 possibly in response to modified elasticity or transparency of the zones 24. The very short laser light pulses cause photomechanical or photoacoustic impacts or shocks illustrated at 26 at the boundaries of the areas 24. The shocks 26 cause localized damage at the boundaries of zones 24b. The second laser light beam may also cause (possibly smaller) shocks in the areas 24b, as illustrated at 26a. The localised damage enhances the release and diffusion of wound healing mediators within and between areas 24 and otherwise unaffected zones 24b, initiating wound healing activity and collagenesis in zones 24b.

Consequently, by subsequently illuminating an area previously treated in a non-ablative manner, with a pulsed laser source, collagen synthesis and skin regeneration over the entire skin area is increased, not simply within or just around the zones 24, leading to an unexpected improvement in the efficacy of the process.

Although in the preceding example, a plurality of zones 24 formed by directing first laser beam 23, and then are subsequently illuminated by second laser beam 25, it will be apparent that this may be performed in any other manner. For example, using the apparatus of FIG. 1a, beam switching device 16 could be operated to direct first laser beam 23 to create an area 24, an then to subsequently direct beam 25 to illuminate the newly created area 24, and then repeat the cycle to create a new area 24 and subsequently re-illuminate the same area. Alternatively, in the example of FIG. 1a, the array of areas 24 may be formed, and then head 14 adjusted or removed to provide a desired width for beam 25. In the example of FIG. 1b, the treatment heads 13a, 13b may be separately adjustable as desired. Further alternatively, the method may be performed using separate apparatuses, one of which has a first laser source 11 and another of which has a second laser source 12.

The apparatus and method described above are suitable for treatment of a range of skin conditions, including the reduction or removal of scars, wrinkles, discoloration and other cosmetic, aesthetic or dermatological issues.

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

Meanings of technical and scientific terms used herein are to be commonly understood as by one of ordinary skill in the art to which the invention belong, unless otherwise defined.

1. A medical apparatus comprising a first laser source operable to generate light at a first wavelength and supply a first laser light beam, a second laser source operable to generate laser light at a second wavelength and supply a second laser light beam, the first laser light beam being continuous or pulsed having a first, relatively long pulse duration and being directed to a first treatment area, the second laser light beam being pulsed with a second, relatively short pulse duration and being directed a second treatment area, the apparatus being operable to supply the first laser light beam and subsequently the second laser light beam.

2. An apparatus according to claim 1, wherein the first laser light beam has a diameter in the range of approximately 100 microns to 400 microns, and preferably in the range 110 to 120 microns.

3. An apparatus according to claim 2 wherein the second laser light beam has a diameter in the range of about 2 to 10 mm, and preferably about 2 to 5 mm.

4. An apparatus according to claim 1 wherein the first wavelength is in the range 1300 to 1600 nm, and preferably about 1565 nm, further preferably such that the first laser beam has a desired coagulative effect.

5. An apparatus according to claim 4 wherein the second wavelength is in the range 500 to 1300 nm.

6. An apparatus according to claim 1 wherein the apparatus is operable to generate the second laser light beam with a pulse length in the range 1 to 100 ns, and preferably less than 10 ns, and most preferably 6 to 8 ns.

7. An apparatus according to claim 1 wherein the apparatus is operable to generate the second laser light beam with second pulse length duration in the range 1 to 700 ps.

8. An apparatus according to claim 1 comprising a treatment head wherein at least the first laser light beam is supplied to the treatment head.

9. An apparatus according to claim 8 wherein the treatment head comprises a scanning device, the scanning device being operable to direct the first laser light beam to a plurality of locations within one or more of the first treatment area and the second treatment area.

10. An apparatus according to claim 1 wherein the first laser source comprises a fibre laser.

11. An apparatus according to claim 1 wherein the second laser source comprises a Q-switched laser.

12. A method of directing laser light to a skin surface, the method comprising the steps of generating a first laser light beam having a first wavelength and direct the first laser light beam to a first irradiation area, subsequently generating a second laser light beam having a second wavelength and directing the second laser light beam to a second irradiation area, the first laser light beam being continuous or pulsed having a first, relatively long pulse duration, the second laser light beam being pulsed with a second, relatively short pulse duration.

13. A method according to claim 12 wherein the method comprises a method of cosmetically treating an area of skin.

14. A method according to claim 12, wherein the first laser light beam has a diameter in the range of approximately 100 microns to 400 microns, and preferably in the range 110 to 120 microns.

15. A method according to claim 14 wherein the second laser light beam has a diameter in the range of about 2 to 10 mm, and preferably in the range 2 to 5 mm.

16. A method according to claim 12 wherein the first wavelength is in the range 1300 nm to 1600 nm, and preferably about 1565 nm, further preferably such that the first laser beam has a desired coagulative effect.

17. A method according to claim 16 wherein the second wavelength is in the range 500 to 1300 nm.

18. A method according to claim 12 wherein the second laser light beam has a pulse length in the range 1 to 100 ns, and preferable less than 10 ns, and most preferable 6 to 8 ns.

19. A method according to claim 12 wherein the second laser light beam has a pulse length in the range 1 to 700 ps.

20. A method according to claim 12 comprising directing the first laser light beam to a plurality of locations within a treatment area.

21. A method according to claim 12 wherein generating the first laser light beam comprises operating a fibre laser.

22. A method according to claim 12 wherein generating the second laser light beam comprises operating a Q-switched laser.

23. The method of claim 12 wherein the first irradiation area and the second irradiation areas are one of: the same, different or overlapping.

24. The apparatus of claim 1 wherein the first and the second treatment areas are one of: the same, different or overlapping.
